(19) United States
(12) Patent Application Publication
(10) Pub. No.: US 2025/0213117 A1
(43) Pub. Date: Jul. 3, 2025

(54) OPTICAL SENSOR DEVICE

(71) Applicant: Taiwan-Asia Semiconductor Corporation, Hsinchu City (TW)

(72) Inventors: Chun-Ko CHEN, Hsinchu City (TW); Cheng-Hsun CHIANG, Hsinchu City (TW)

(21) Appl. No.: 18/735,345

(22) Filed: Jun. 6, 2024

(30) Foreign Application Priority Data
Dec. 28, 2023 (TW) 112151341

(51) Int. Cl. A61B 5/00

(52) U.S. Cl. CPC A61B 5/0059; A61B 2560/0431; A61B 2562/0238

(57) ABSTRACT

An optical sensor device is provided. The optical sensor device includes a carrier substrate, a plurality of light sources disposed on the carrier substrate for generating light of a plurality of wavelengths, a photodiode sensor disposed on the carrier substrate and spaced apart from the light sources at a distance, and a multi-passband filter formed on a top surface of the photodiode sensor. The multi-passband filter has a plurality of passbands corresponding to the light of the plurality of wavelengths from the light sources.

### CROSS-REFERENCES TO RELATED APPLICATIONS

This application claims priority to Taiwan Patent Application No. 112151341 filed on Dec. 28, 2023, which is hereby incorporated by reference in its entirety.

### BACKGROUND OF THE INVENTION

### Field of the Invention

The present invention relates to an optical sensor device, particularly an optical sensor device with a multi-passband filter applied to a wearable device or a handheld device.

### Descriptions of the Related Art

In recent years, non-invasive optical sensor devices, especially wearable or handheld ones, have been widely used in people's daily lives for purposes such as activity tracking, health management, and disease detection. These devices provide users with various physiological information, e.g., heart rates, blood oxygen saturation, blood pressure, and blood glucose, etc.

To obtain various physiological information, optical sensor devices in the prior art require a plurality of light sources to generate light of different target wavelengths (e.g., green light, red light and near-infrared light, with wavelengths ranging from 300 nm to 1100 nm), and are equipped with a plurality of optical sensors (e.g., photodiode sensors) to receive light of different target wavelengths. Typically, the green light is used to measure heart rates, while the red light and the near-infrared light are used to measure blood oxygen saturation.

In addition, light sources with wavelengths ranging from 300 nm to 1100 nm are also applied to camera modules in mobile phones in practice. The ambient light sensors of the camera modules also require a plurality of light sources to generate light of different target wavelengths and a plurality of light sensors to receive light of different target wavelengths. The ambient light sensor detects the physical flicker of ambient light (light generated by 3C devices, such as TVs, computers and mobile phones) to provide image correction input. This helps eliminate the stripes and artifacts caused by ambient light flicker so as to prevent distortion in captured images and/or videos.

Under the structure of this conventional technology, the optical sensor device cannot only use a single optical sensor to receive all the light, which will cause the ambient light of 350-800 nm to be regarded as noise (e.g., light generated by daylight, indoor lighting and 3C devices) is difficult to be excluded. Secondly, the responsivities of a single optical sensor are different for green light, red light, and near-infrared light. Green light has the weakest responsivity, while near-infrared light has the strongest with a gradient increase. This results in significant differences in the responsivities of the three light sources. Therefore, it is difficult for optical sensor devices to process light with different responsivities using only a single optical sensor to define the measurement values for different wavelengths.

Accordingly, it is impossible to reduce the number of optical sensors required to be used in the optical sensor device by the conventional technology, and therefore, it is difficult to further miniaturize the optical sensor device and reduce the manufacturing cost.

Given this, how to reduce the number of optical sensors used in the optical sensor device, further miniaturize the optical sensor device and reduce the manufacturing cost is an urgent issue for the industry to solve.

### SUMMARY OF THE INVENTION

An objective of the present invention is to use only a single optical sensor in an optical sensor device for further miniaturizing the optical sensor device and reducing manufacturing costs. The present invention introduces optical coating technology and designs a gradient filter that can filter three or four wavelengths at the same time to define the receiving wavelengths of the optical sensor. And, the receiving intensity for some wavelengths can be reduced according to the design requirements, e.g., the received light percentage of the green light is the highest, followed by red light, and near-infrared light is the lowest. Therefore, by adding a multi-passband filter, the present invention not only eliminates noise light other than the target wavelength required for measurement but also allows a single optical sensor to have the same responsivity for different target wavelengths through the design of the present invention.

To achieve the above objective, the present invention discloses an optical sensor device including: a carrier substrate for generating light of a plurality of wavelengths, a plurality of light sources disposed on the carrier substrate; a photodiode sensor disposed on the carrier substrate and spaced apart from the light sources at a distance; and a multi-passband filter formed on a top surface of the photodiode sensor, wherein the multi-passband filter has a plurality of passbands corresponding to the light of the plurality of wavelengths from the light sources.

In an example, the wavelengths include a first wavelength, a second wavelength and a third wavelength, and the first wavelength, the second wavelength and the third wavelength are different from each other and between 300-1000 nm.

In an example, the passbands include a first passband corresponding to the first wavelength, a second passband corresponding to the second wavelength, and a third passband corresponding to the third wavelength. Light transmittance of each of the first passband, the second passband and the third passband is between 25%-98%, and full width at half maximum (FWHM) of each of the first passband, the second passband and the third passband is between 30-80 nm.

In an example, the third wavelength is greater than the second wavelength, and the second wavelength is greater than the first wavelength. The light transmittance of the third passband is at least 5% less than the light transmittance of the second passband, and the light transmittance of the second passband is at least 5% less than the light transmittance of the first passband.

In an example, the first wavelength is 525 nm, the second wavelength is 660 nm, and the third wavelength is 850 nm.

In an example, the wavelengths include a first wavelength, a second wavelength, a third wavelength and a fourth wavelength, and the first wavelength, the second wavelength, the third wavelength and the fourth wavelength are different from each other and between 300-1000 nm.

In an example, the passbands include a first passband corresponding to the first wavelength, a second passband corresponding to the second wavelength, a third passband corresponding to the third wavelength and the fourth passband corresponding to the fourth wavelength. Light transmittance of each of the first passband, the second passband, the third passband and the fourth passband is between 25%-98%, and FWHM of each of the first passband, the second passband, the third passband and the fourth passband is between 30-80 nm.

In an example, the fourth wavelength is greater than the third wavelength, the third wavelength is greater than the second wavelength, and the second wavelength is greater than the first wavelength. The light transmittance of the fourth passband is at least 5% less than the light transmittance of the third passband, the light transmittance of the third passband is at least 5% less than the light transmittance of the second passband, and the light transmittance of the second passband is at least 5% less than the light transmittance of the first passband.

In an example, the first wavelength is 525 nm, the second wavelength is 660 nm, the third wavelength is 850 nm, and the fourth wavelength is 940 nm.

In an example, the multi-passband filter is formed by first dielectric material layers and second dielectric material layers alternately stacked to form a multilayer structure. Each of the first dielectric material layers is composed of one of tantalum pentoxide (Ta₂O₅) and titanium dioxide (TiO₂), and each of the second dielectric material layers is composed of one of silicon dioxide (SiO₂) and aluminum oxide (Al₂O₃).

In an example, the multilayer structure further includes an aluminum layer between two of the first dielectric material layers.

In an example, the light sources are a plurality of light-emitting diodes (LEDs).

In an example, the optical sensor device is used in a wearable device.

In an example, the optical sensor device is used in a handheld device.

After referring to the drawings and the detailed description of embodiments described later, those of ordinary skill in the art can understand other objectives of the present invention, as well as the technical means and implementations of the present invention.

### BRIEF DESCRIPTION OF THE DRAWINGS

FIG. 1A is a schematic top view of an optical sensor device according to an embodiment;

FIG. 1B is a schematic side view of the optical sensor device of FIG. 1A;

FIG. 2A is a schematic top view of an optical sensor device according to another embodiment;

FIG. 2B is a schematic side view of the optical sensor device of FIG. 2A;

FIGS. 3A to 3E illustrate the production of a photodiode sensor and a multi-passband filter according to an embodiment;

FIG. 4 shows the transmittance and the full width at half maximum (FWHM) of each of the first passband, the second passband, the third passband and the fourth passband of the multi-passband filter; and

FIG. 5 shows the relative responsivities of the photodiode sensor to light of each wavelength before adding the multi-passband filter, and the relative responsivities of the photodiode detector to the light of the first wavelength, the second wavelength, the third wavelength and the fourth wavelength after adding the multi-passband filter according to an embodiment.

### DETAILED DESCRIPTION OF THE PREFERRED EMBODIMENT

In the following description, the present invention will be explained with reference to various embodiments thereof. These embodiments of the present invention are not intended to limit the present invention to any specific environment, application or particular method for implementations described in these embodiments. Therefore, the description of these embodiments is for illustrative purposes only and is not intended to limit the present invention. It shall be appreciated that, in the following embodiments and the attached drawings, partial elements not directly related to the present invention are omitted from the illustration, and dimensional proportions among individual elements and the numbers of each element in the accompanying drawings are provided only for ease of understanding but are not intended to limit the actual scale.

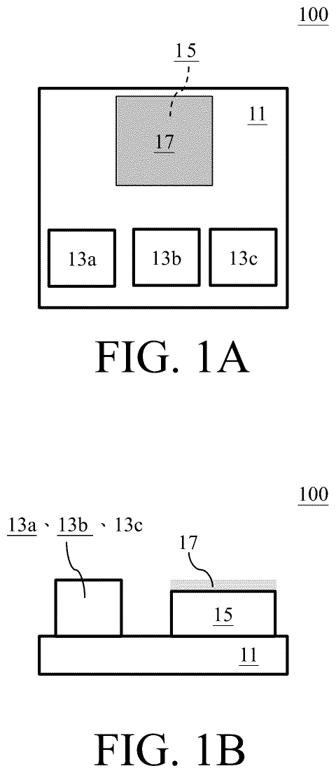

An embodiment of the present invention is shown in FIG. 1A and FIG. 1B. FIG. 1A is a schematic top view of an optical sensor device 100. FIG. 1B is a schematic side view of the optical sensor device 100.

The optical sensor device 100 includes a carrier substrate 11, a plurality of light sources 13a, 13b, 13c, a photodiode sensor 15 and a multi-passband filter 17.

In this embodiment, the optical sensor device 100 has three light sources 13a, 13b and 13c as an example. However, in other examples, the optical sensor device may have two light sources or more than three light sources. The light sources 13a, 13b and 13c are provided on the carrier substrate 11, respectively. The light sources 13a, 13b and 13c are a plurality of light-emitting diodes (LEDs) that generate light of different target wavelengths. For example, the wavelengths (i.e., the first wavelength, the second wavelength, and the third wavelength) generated by the light sources 13a, 13b and 13c range from 300 nm to 1000 nm. For example, the wavelength of the light generated by the light source 13a (i.e., the first wavelength) is 525 nm, the wavelength of the light generated by the light source 13b (i.e., the second wavelength) is 660 nm, and the wavelength of the light generated by the light source 13c (i.e., the third wavelength) is 850 nm.

The photodiode sensor 15 is also disposed on the carrier substrate 11 and is spaced apart from the light sources 13a, 13b and 13c at a distance. The photodiode sensor 15 may be an indium gallium arsenide (InGaAs) photodiode sensor, but is not limited thereto. The photodiode sensor 15 may detect light in a wavelength range of 300 nm to 1100 nm, but is not limited thereto, and may vary depending on different applications.

The multi-passband filter 17 is formed on a top surface of the photodiode sensor 15. The multi-passband filter 17 has a plurality of passbands corresponding to the light of the wavelengths (i.e., the first wavelength, the second wavelength and the third wavelength) of the light sources 13a, 13b and 13c. For example, the passbands include a first passband corresponding to the first wavelength (e.g., 525 nm), a second passband corresponding to the second wavelength (e.g., 660 nm) and a third passband corresponding to the third wavelength (e.g., 850 nm).

Furthermore, the multi-passband filter 17 may be composed of first dielectric material layers and second dielectric material layers alternately stacked to form a multilayer structure. For example, the first dielectric material layer may use a material with a higher refractive index, e.g., it may be composed of one of tantalum pentoxide (Ta₂O₅) and titanium dioxide (TiO₂). The second dielectric material layer may use a material with a lower refractive index, e.g., it may be composed of one of silicon dioxide (SiO₂) and aluminum oxide (Al₂O₃). The refractive index of the first dielectric material layer and the second dielectric material layer is between 1.4 and 3. For example, the refractive index of Ta₂O₅ is 2.1, that of TiO₂ is 2.4, that of SiO₂ is 1.48, and that of Al₂O₃ is 1.64. The multilayer structure may contain 40 to 70 layers, with the total thickness ranging from 3 μm to 10 μm, but the number of layers and the total thickness may vary depending on different applications.

In addition, in an embodiment, the multilayer structure may further include an aluminum layer between two of the first dielectric material layers. Aluminum is a material with a very low refractive index and its refractive index is 1.2. Therefore, in the embodiment of the present invention, the number of layers and thickness of the required multilayer structure can be reduced by adding an aluminum layer to the multilayer structure.

It should be noted that in FIGS. 1A and 1B, the positions of the light sources 13a, 13b, 13c and the photodiode sensors 15 arranged on the carrier substrate 11 of the optical sensor device 100 are depicted for illustrative purposes only. For those skilled in the art, it is generally understood that the positions of the light sources 13a, 13b, 13c, and photodiode sensors 15 may vary depending on the specific application. Therefore, the positions depicted in FIGS. 1A and 1B are not intended to limit the scope of the present invention. Moreover, for simplicity, other components of the optical sensor device 100, such as a glass carrier, a sealing layer, etc., are not shown in the drawings and will not be further described herein.

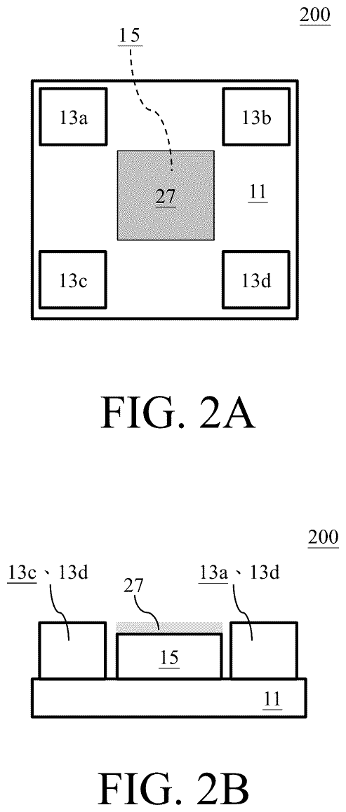

Another embodiment of the present invention is shown in FIG. 2A and FIG. 2B. FIG. 2A is a schematic top view of an optical sensor device 200. FIG. 2B is a schematic side view of the optical sensor device 200. Different from the optical sensor device 100, in this embodiment, the optical sensor device 200 has four light sources 13a, 13b, 13c and 13d as an example. The light sources 13a, 13b, 13c and 13d are provided on the carrier substrate 11, respectively. The light sources 13a, 13b, 13c and 13d are a plurality of light-emitting diodes (LEDs) that generate light of different target wavelengths. For example, the wavelengths (i.e., the first wavelength, the second wavelength, the third wavelength and the fourth wavelength) generated by the light sources 13a, 13b, 13c and 13d range from 300 nm to 1000 nm. For example, the wavelength of the light generated by the light source 13a (i.e., the first wavelength) is 525 nm, the wavelength of the light generated by the light source 13b (i.e., the second wavelength) is 660 nm, the wavelength of the light generated by the light source 13c (i.e., the third wavelength) is 850 nm, and the wavelength of the light generated by the light source 13d (i.e., the fourth wavelength) is 940 nm.

In addition, in this embodiment, different from the multi-passband filter 17 of the first embodiment, the multi-passband filter 27 has a plurality of passbands corresponding to the wavelengths (i.e., the first wavelength, the second wavelength, the third wavelength and the fourth wavelength) of the light sources 13a, 13b, 13c and 13d. For example, the passbands include a first passband corresponding to the first wavelength (e.g., 525 nm), a second passband corresponding to the second wavelength (e.g., 660 nm), the third passband corresponding to the third wavelength (e.g., 850 nm), and the fourth passband corresponding to the fourth wavelength (e.g., 940 nm).

It should be noted that in FIGS. 2A and 2B, the positions of the light sources 13a, 13b, 13c, 13d and the photodiode sensors 15 arranged on the carrier substrate 11 of the optical sensor device 200 are depicted for illustrative purposes only. For those skilled in the art, it is generally understood that the positions of the light sources 13a, 13b, 13c, 13d and photodiode sensors 15 may vary depending on the specific application. Therefore, the positions depicted in FIGS. 2A and 2B are not intended to limit the scope of the present invention. Likewise, for simplicity, other components of the optical sensor device 200, such as a glass carrier, a sealing layer, etc., are not shown in the drawings and will not be further described herein.

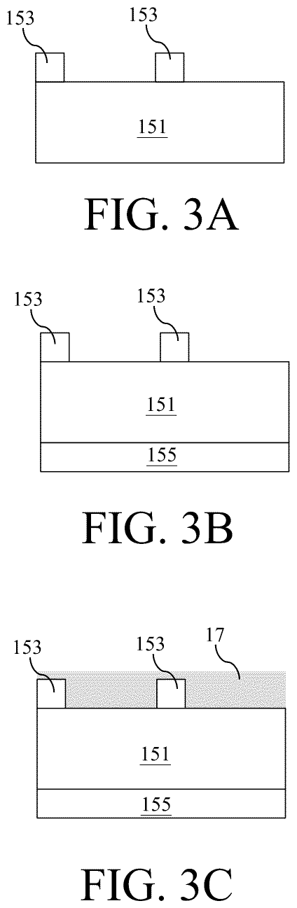

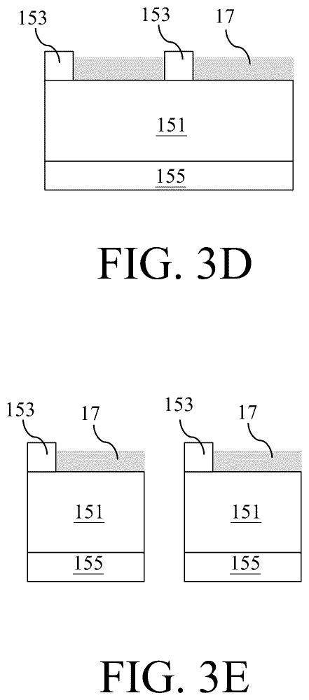

An embodiment of the present invention is shown in FIGS. 3A to 3E, which illustrate the production of the photodiode sensor 15 and the multi-passband filter 17 (or the multi-passband filter 27). To simplify the description, only the multi-passband filter 17 is depicted in FIGS. 3A to 3E as an example. First, as shown in FIG. 3A, a photodiode wafer 151 is provided, and a plurality of upper electrodes 153 are formed on the photodiode wafer 151 (due to the layout constraint of the drawing, only two upper electrodes 153 are depicted for illustration). Next, as shown in FIG. 3B, a lower electrode 155 is formed under the photodiode wafer 151, and as shown in FIG. 3C, a multi-passband filter 17 is coated on the photodiode wafer 151 and the upper electrodes 153.

Subsequently, the multi-passband filters 17 on the upper electrodes 153 are removed (e.g., using a yellow-light photolithography process), as shown in FIG. 3D. Finally, dicing is performed to form a plurality of photodiode sensors 15 with the multi-passband filters 17 coated on top.

It should be noted that FIGS. 3A to 3E are only an example for illustrating a method of manufacturing the photodiode sensor 15 and the multi-passband filter 17. In other words, in practice, there may be other alternatives to the order of the manufacturing method and to the process to be used. Therefore, the manufacturing method of the photodiode sensor 15 and the multi-passband filter 17 of the present invention is not limited to the steps shown in FIGS. 3A to 3E.

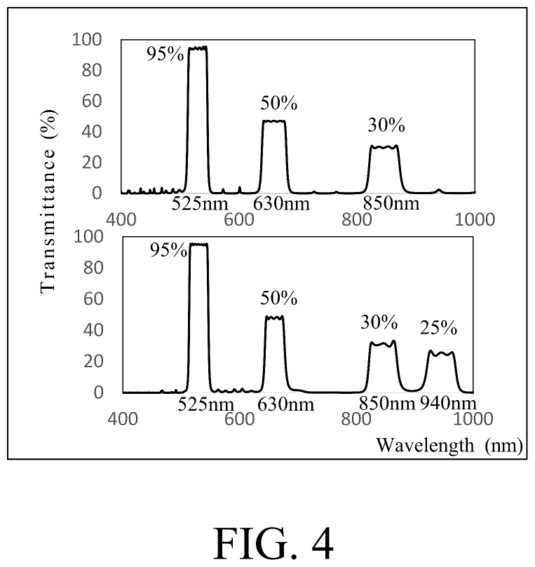

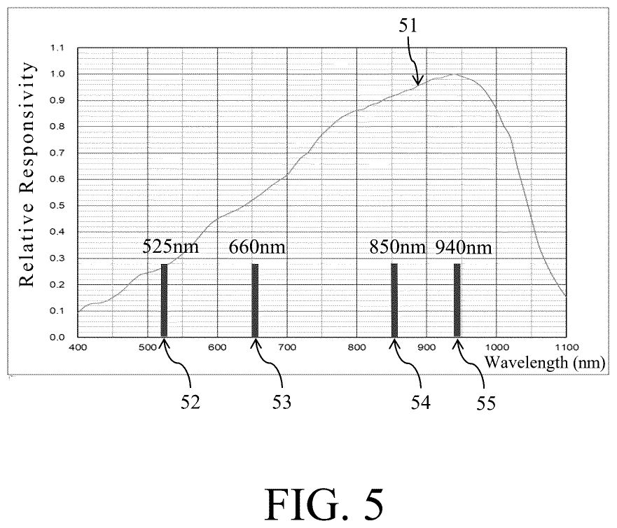

Please refer to FIG. 4 and FIG. 5 for an embodiment of the present invention. As shown in FIG. 4, the light transmittance of each of the first passband (corresponding to the first wavelength), the second passband (corresponding to the second wavelength) and the third passband (corresponding to the third wavelength) of the multi-passband filter 17 ranges from 25% to 98%, and the FWHM of each of the first passband, the second passband and the third passband ranges from 30 nm to 80 nm.

In addition, as mentioned above, the third wavelength (e.g., 850 nm) is greater than the second wavelength (e.g., 660 nm) and the second wavelength is greater than the first wavelength (e.g., 525 nm). In the present invention, to make the photodiode sensor 15 have the same responsivity for different target wavelengths, the light transmittance of the third passband is at least 5% less than the light transmittance of the second passband and the light transmittance of the second passband is at least 5% less than the light transmittance of the first passband. For example, as shown in FIG. 4, the light transmittance of the third passband is 30%, the light transmittance of the second passband is 50%, and the light transmittance of the first passband is 95%.

Similarly, as shown in FIG. 4, the light transmittance of each of the first passband (corresponding to the first wavelength), the second passband (corresponding to the second wavelength), the third passband (corresponding to the third wavelength) and the four passbands (corresponding to the fourth wavelength) of the multi-passband filter 27 ranges from 25% to 98%, and the FWHM of each of the first passband, the second passband, the third passband and the fourth passband ranges from 30 nm to 80 nm.

Likewise, as mentioned above, the fourth wavelength (e.g., 940 nm) is greater than the third wavelength (e.g., 850 nm), the third wavelength is greater than the second wavelength (e.g., 660 nm), and the second wavelength is greater than the first wavelength (e.g., 525 nm). In the present invention, to make the photodiode sensor 15 have the same responsivity for different target wavelengths, the light transmittance of the fourth passband is at least 5% less than the light transmittance of the third passband, the light transmittance of the third passband is at least 5% less than the light transmittance of the second passband, and the light transmittance of the second passband is at least 5% less than the light transmittance of the first passband. For example, as shown in FIG. 4, the light transmittance of the fourth passband is 25%, the light transmittance of the third passband is 30%, the light transmittance of the second passband is 50%, and the light transmittance of the first passband is 95%.

FIG. 5 shows the relative responsivities of the photodiode sensor 15 for each wavelength of light before adding the multi-passband filter 27 (the curve 51 in FIG. 5) and the relative responsivities of the photodiode sensor 15 for the light of the first wavelength (e.g., 525 nm), the second wavelength (e.g., 660 nm), the third wavelength (e.g., 850 nm) and the fourth wavelength (e.g., 940 nm) after adding the multi-passband filter 27 (the bar lines 52, 53, 54 and 55 in FIG. 5). From FIG. 5, it can be seen that by adding the multi-passband filter 27 to the photodiode sensor 15, the present invention enables the photodiode sensor 15 to have the same responsivity for the first, second, third, and fourth wavelengths. Therefore, by adding the multi-passband filter 17 or the multi-passband filter 27, the present invention effectively eliminates noise light outside the target wavelengths required for measurement and ensures that the photodiode sensor 15 has the same responsivity for different target wavelengths.

In practical applications, the optical sensor devices 100 and 200 of the present invention may be used in a wearable device (e.g., a smart watch, a smart bracelet or any wearable device that can be worn on the human body). In addition, in practical applications, the optical sensor devices 100 and 200 of the present invention may also be used in a handheld device (e.g., a camera, a camera module of a mobile phone, or any handheld device with a camera module).

In summary, the optical sensor device of the present invention only uses a single photodiode sensor, so it can effectively further miniaturize the optical sensor device and reduce manufacturing costs. Furthermore, the present invention introduces optical coating technology and designs a multi-passband filter that can filter three or four wavelengths at the same time to define the receiving wavelength of the optical sensor and allow the receiving intensity to be reduced according to design requirements. Therefore, by adding a multi-passband filter, the present invention not only eliminates noise light outside the target wavelength required for measurement but also allows a single photodiode sensor to have the same responsivity for different target wavelengths through the design of the present invention.

The above embodiments are used only to illustrate the implementations of the present invention and to explain the technical features of the present invention, and are not intended to limit the scope of the present invention. Any modifications or equivalent arrangements that can be easily accomplished by those skilled in this art are considered to fall within the scope of the present invention, and the scope of the present invention should be limited by the claims of the patent application.

What is claimed is:

1. An optical sensor device, comprising
a carrier substrate;
a plurality of light sources disposed on the carrier substrate for generating light of a plurality of wavelengths;
a photodiode sensor disposed on the carrier substrate and spaced apart from the light sources at a distance; and
a multi-passband filter formed on a top surface of the photodiode sensor, wherein the multi-passband filter has a plurality of passbands corresponding to the light of the plurality of wavelengths from the light sources.

2. The optical sensor device of claim 1, wherein the wavelengths comprise a first wavelength, a second wavelength and a third wavelength, and the first wavelength, the second wavelength and the third wavelength are different from each other and between 300-1000 nm.

3. The optical sensor device of claim 2, wherein the passbands comprise a first passband corresponding to the first wavelength, a second passband corresponding to the second wavelength, and a third passband corresponding to the third wavelength, light transmittance of each of the first passband, the second passband and the third passband is between 25%-98%, and full width at half maximum (FWHM) of each of the first passband, the second passband and the third passband is between 30-80 nm.

4. The optical sensor device of claim 3, wherein the third wavelength is greater than the second wavelength, and the second wavelength is greater than the first wavelength, and wherein the light transmittance of the third passband is at least 5% less than the light transmittance of the second passband, and the light transmittance of the second passband is at least 5% less than the light transmittance of the first passband.

5. The optical sensor device of claim 3, wherein the first wavelength is 525 nm, the second wavelength is 660 nm, and the third wavelength is 850 nm.

6. The optical sensor device of claim 1, wherein the wavelengths comprise a first wavelength, a second wavelength, a third wavelength and a fourth wavelength, and the first wavelength, the second wavelength, the third wavelength and the fourth wavelength are different from each other and between 300-1000 nm.

7. The optical sensor device of claim 6, wherein the passbands comprise a first passband corresponding to the first wavelength, a second passband corresponding to the second wavelength, a third passband corresponding to the third wavelength and the fourth passband corresponding to the fourth wavelength, light transmittance of each of the first passband, the second passband, the third passband and the fourth passband is between 25%-98%, and FWHM of each of the first passband, the second passband, the third passband and the fourth passband is between 30-80 nm.

8. The optical sensor device of claim 7, wherein the fourth wavelength is greater than the third wavelength, the third wavelength is greater than the second wavelength, and the second wavelength is greater than the first wavelength, and wherein the light transmittance of the fourth passband is at least 5% less than the light transmittance of the third passband, the light transmittance of the third passband is at least 5% less than the light transmittance of the second passband, and the light transmittance of the second passband is at least 5% less than the light transmittance of the first passband.

9. The optical sensor device of claim 6, wherein the first wavelength is 525 nm, the second wavelength is 660 nm, the third wavelength is 850 nm, and the fourth wavelength is 940 nm.

10. The optical sensor device of claim 1, wherein the multi-passband filter is formed by first dielectric material layers and second dielectric material layers alternately stacked to form a multilayer structure, each of the first dielectric material layers is composed of one of tantalum pentoxide (Ta₂O₅) and titanium dioxide (TiO₂), and each of the second dielectric material layers is composed of one of silicon dioxide (SiO₂) and aluminum oxide (Al₂O₃).

11. The optical sensor device of claim 10, wherein the multilayer structure further comprises an aluminum layer between two of the first dielectric material layers.

12. The optical sensor device of claim 1, wherein the light sources are a plurality of light-emitting diodes (LEDs).

13. The optical sensor device of claim 1, wherein the optical sensor device is used in a wearable device.

14. The optical sensor device of claim 1, wherein the optical sensor device is used in a handheld device.
